(19) United States
(12) Patent Application Publication
(10) Pub. No.: US 2025/0005678 A1
(43) Pub. Date: Jan. 2, 2025

(54) SYSTEMS AND METHODS FOR LAPSE DETECTION

(71) Applicant: PointCare, LLC., Walnut Creek, CA (US)

(72) Inventors: Everett Lebherz, Alamo, CA (US); Eric Zingeler, Pinole, CA (US); Bart Collins, Fort Collins, CO (US)

(21) Appl. No.: 18/751,534

(22) Filed: Jun. 24, 2024

Related U.S. Application Data
(60) Provisional application No. 63/511,418, filed on Jun. 30, 2023.

(51) Int. Cl. G06Q 40/08; G06Q 10/109; G16H 10/60

(52) U.S. Cl. CPC G06Q 40/08; G06Q 10/109; G16H 10/60

(57) ABSTRACT

A method for lapse detection includes receiving, from a database associated with a healthcare insurer, a first record associated with a patient and the healthcare insurer during a previous time period, and receiving, from the database, a second record associated with the patient and the healthcare insurer during a current time period. The method further includes detecting an enrollment lapse for the patient based on the first record and the second record, and generating a notification to the patient to inform the patient of the enrollment lapse.

### CROSS-REFERENCE

The application claims priority to U.S. provisional patent application No. 63/511,418, filed Jun. 30, 2023, the contents of which are incorporated herein in their entirety.

### BACKGROUND

### 1. Technical Field

The disclosure relates to healthcare technology, specifically related to health insurance enrollment and lapse detection.

### 2. Discussion of Related Art

Historically, healthcare providers have used traditional methods of enrollment and lapse detection, such as manual tracking of patient coverage status, to enroll patients in public coverage and detect lapses in coverage. This process can be time-consuming and error-prone, and it relies heavily on individual provider initiative to track patients' coverage status. As a result, lapses in coverage may go undetected, leading to uncovered visits and decreased continuity of patient care.

Many states have their own Medicaid enrollment websites that allow patients to enroll in public coverage online. However, these websites can be difficult to navigate, and patients may need assistance in completing the enrollment process. This can lead to delays in coverage and decreased patient satisfaction.

Enrollment brokers are third-party organizations that help patients enroll in public coverage. However, these brokers may charge fees for their services, which can be a barrier to patients who cannot afford them. In addition, they may not be available in all areas, making it difficult for patients in rural or underserved areas to access their services.

Some healthcare providers use automated systems to verify patient eligibility for public coverage. These systems can assist in detecting lapses in coverage (through other means as discussed above), but they do not provide proactive outreach to assist patients with re-enrollment. In addition, these systems may be expensive to implement and maintain.

Each of these prior approaches has failed to fully address the challenges associated with enrolling patients in public coverage and detecting lapses in coverage. These prior approaches also use extraneous elements that can add complexity and cost to the enrollment process. For example, enrollment brokers add an additional layer of bureaucracy and may charge fees for their services, while automated systems for eligibility verification require significant investment in software and infrastructure.

### SUMMARY

According to various embodiments, a method for lapse detection includes receiving, from a database associated with a healthcare insurer, a first record associated with a patient and the healthcare insurer during a previous time period, and receiving, from the database, a second record associated with the patient and the healthcare insurer during a current time period. The method further includes detecting an enrollment lapse for the patient based on the first record and the second record, and generating a notification to the patient to inform the patient of the enrollment lapse.

According to some embodiments, a non-transitory computer-readable medium stores a set of instructions for lapse detection, which when executed by a computer, configure the computer to receive, from a database associated with a healthcare insurer, a first record associated with a patient and the healthcare insurer during a previous time period, and receive, from the database, a second record associated with the patient and the healthcare insurer during a current time period. The instructions, when executed by the computer, further configure the computer to detect an enrollment lapse for the patient based on the first record and the second record, and generate a notification to the patient to inform the patient of the enrollment lapse.

### BRIEF DESCRIPTION OF THE DRAWINGS

Further objectives and advantages will become apparent from a consideration of the description, drawings, and examples.

FIG. 1 illustrates an example of a system configured to perform lapse detection;

FIG. 2 illustrates an example of operations performed in some embodiments by the system of FIG. 1;

FIG. 3 illustrates an example method embodiment for lapse detection;

FIG. 4 illustrates an example of communications between a provider, a clearinghouse, and providers;

FIG. 5 illustrates an example of a system communicating with a clearinghouse; and

FIG. 6 illustrates an example computer system.

### DETAILED DESCRIPTION

Some embodiments of the disclosure are discussed in detail below. In describing embodiments, specific terminology is employed for the sake of clarity. However, the disclosure is not intended to be limited to the specific terminology so selected. A person skilled in the relevant art will recognize that other equivalent components can be employed, and other methods developed, without departing from the broad concepts of the disclosure. All references cited anywhere in this specification, including the Background and Detailed Description sections, are incorporated by reference as if each had been individually incorporated.

Systems and methods as disclosed herein identify when lapses in insurance coverage occur for individuals, allowing those individuals to be notified. The system operates by requesting (on a periodic basis or upon detection of a triggering event) a list of those with coverage in a current period, as well as those who had coverage in a previous period. For example, the system can send a request (such as, but not limited to a Representational State Transfer (REST) request; a JavaScript Object Notation (JSON) request; a Hypertext Transfer Protocol (HTTP); a POST request; or some combination thereof) to a clearinghouse for a single patient. This request can include the patient's identified information (e.g., first name, last name, date of birth, SSN, insurance subscriber ID) and date of service. The clearinghouse can translate this request into an “EDI 270” (Electronic Data Interchange 270) transaction and send it to the target payer. The payer can process the EDI 270 request and returns an EDI 271 response with eligibility and benefit information. The clearinghouse can then convert the EDI 271 response back into a JSON format (or other desired format) and send it back to the system via a REST response. The data transmitted back to the system can include data about one patient, or data about many patients. For example, the requested data could be of everyone that has coverage this month, as well as those that had coverage last month (please note that the time period may change depending on the specific configuration). The request is sent from the system to one or more insurance clearing houses. A clearing house is an entity which operates, on behalf of coverage providers (e.g., doctors, dentists, etc.) to help with communicating with various insurance providers. Non-limiting examples of such insurance providers can include BLUE CROSS BLUE SHIELD, as well as semi-public providers such as MEDICARE, and MEDICAID.

The clearing house can then forward the request to the different providers, receiving information about those covered in the current time period (e.g., this month) as well as those that were covered in the previous time period (e.g., last month). However, in many instances, the distinct providers provide the data in distinct formats. The system can parse and/or normalize the data as needed, resulting in normalized data. For example, the source data can be extracted from the clearing house response (e.g., an EDI 271 response, as previously outlined). For each payor that the system integrates from different payors (e.g., MEDI-CAL, Texas MEDICAID), the system can have a specialized data parser to determine the patient's coverage status, whether active or inactive, based on the eligibility benefits data within the 271 response. Each payor can supply an “Eligibility 270/271 Companion Guide,” which specifies the benefits and requirements. This guide serves as the foundational reference for developing the payor-specific parser, enabling the translation of identifiers into meaningful information.

The benefits information can include a list of current benefits the patient is eligible for (e.g., emergency care, outpatient services, inpatient care, prescription drugs), along with any plans they are currently enrolled in. Generally, a patient is considered active if they are eligible to receive any inpatient or outpatient services from the payor in question. The system can then identify lapses in coverage for individuals using the normalized data, where (for example) someone had coverage last month but does not have coverage this month. The system can then generate notifications for those individuals and/or their providers, then send those notifications to the individuals and/or providers such that the individuals do not unintentionally lack insurance. Notifications can be generated, for example, based on predefined rules in batch scripts (such as, but not limited to, Structured Query Language (SQL) batch scripts) within the system's data pipeline. Data can be extracted from a transactional database storing the 270/271 data and loaded into the system's SQL data warehouse. Scheduled SQL batch scripts can then determine the current state of each member, identifying whether they are active, inactive, or have lapsed. If a member is newly lapsed and has not yet received a notification, a notification is queued for that member in a SQL table. The queued notifications can then be sent out manually or automatically, depending upon configuration. The content of the notifications can be generated using predefined templates. Preferably, the notifications are sent to the healthcare providers, informing them that they have patients who have recently lapsed.

In various embodiments, a “lapse” may refer to healthcare insurance coverage for a patient which was previously had an active enrollment status but has now changed to an inactive enrollment status. The healthcare insurance coverage may be provided to the patient by a healthcare insurer.

In various embodiments, “healthcare insurer” may refer to an entity that provides medical health insurance to a patient. The entity may be a private entity such as (but not limited to) BLUE CROSS BLUE SHIELD, or may be a public entity such as (but not limited to) MEDICARE and MEDICAID.

In various embodiments, “enrollment status” may refer to the status of the patient's healthcare coverage with a healthcare insurer. The enrollment status may include a status such as “active” or “inactive,” as well as a date or range of dates of coverage. The enrollment status of a patient may be obtained by making a query to a database of the healthcare insurer, such as a Healthcare Eligibility, Coverage, and Benefit Inquiry (also referred to as a “270 Request”). The enrollment status may be received from the healthcare insurer as a reply to the query, such as a Healthcare Eligibility, Coverage, and Benefit Response (also referred to as a “271 Response”). The query and response to the healthcare insurer may be performed using an Application Programming Interface (API) or an Electronic Data Interchange (EDI) transaction. The enrollment status of the patient may be received in a proprietary data format that is specific to an entity such as (but limited to) a healthcare insurer, a medical organization, and a government entity. Alternatively, the enrollment status of the patient may be received in a common data format that is not specific to any entity. The enrollment status of the patient may be converted or transformed between formats.

In various embodiments, “trigger event” may refer to an event that is associated with a patient's health care coverage or treatment. For example, the trigger event may be a change in enrollment status or eligibility, including but not limited to changes in household (e.g., getting married or divorced, births, deaths), changes in residence (e.g., moving to/from a different country), and changes in status (e.g., becoming a citizen). Alternatively, the trigger event may be receiving healthcare services, such as having labs taken or ordered, making an appointment with a medical provider, etc.

In some embodiments, “medical provider” may refer to a medical organization that provides medical services, facilities, or resources to patients or medical providers. Examples of such medical providers include, but are not limited to, hospitals, clinics, medical practices, research institutions, corporations, and other entities involved in the development, delivery, or management of medical care.

In various embodiments, “medical provider” may refer to a medical professional that is capable of providing a treatment to a patient. Examples of such medical providers include, but are not limited to, physicians, nurses, pharmacists, dentists, and other types of licensed health professionals and health practitioners. A medical provider may have a relationship with a medical organization, including but not limited to being an employee, being an owner, being a resident or a student, being a partner, or having attending privileges therewith.

Some embodiments provide a cloud-based platform that healthcare providers can use to manage and track patient coverage status, including notifications of lapses in coverage, and to provide assistance with enrollment.

Some embodiments provide a device or software application that integrates with electronic medical records and automatically detects and flags patients who may be eligible for public coverage based on their income or other criteria. Some embodiments work with different public coverage programs, such as Medicare or state-based programs.

Some embodiments integrate with other healthcare technologies, including but not limited to electronic health records or patient portals, to streamline the enrollment and re-enrollment process. Some embodiments have potential commercial applications in the healthcare industry, specifically in the field of public management. Some embodiments may be implemented in different healthcare settings, including but not limited to clinics, hospitals, or community health centers.

Some embodiments are adapted for different patient populations, such as those with limited English proficiency or those with disabilities, to ensure equitable access to public coverage. Some embodiments are adapted for different geographic areas, such as urban or rural settings, to address geographical variations in healthcare access and enrollment.

Some embodiments provide a number of advantages, including but not limited to increasing patient enrollment in public coverage by providing an easier and more streamlined enrollment process; detecting lapses in public coverage and providing automated ways to outreach to patients to assist with re-enrollment, particularly by text message; improving patient care outcomes by reducing the number of uncovered visits and ensuring continuity of patient care; reducing costs associated with uncovered visits for healthcare providers; and enhancing the patient experience by providing a more seamless and convenient public coverage enrollment and re-enrollment process.

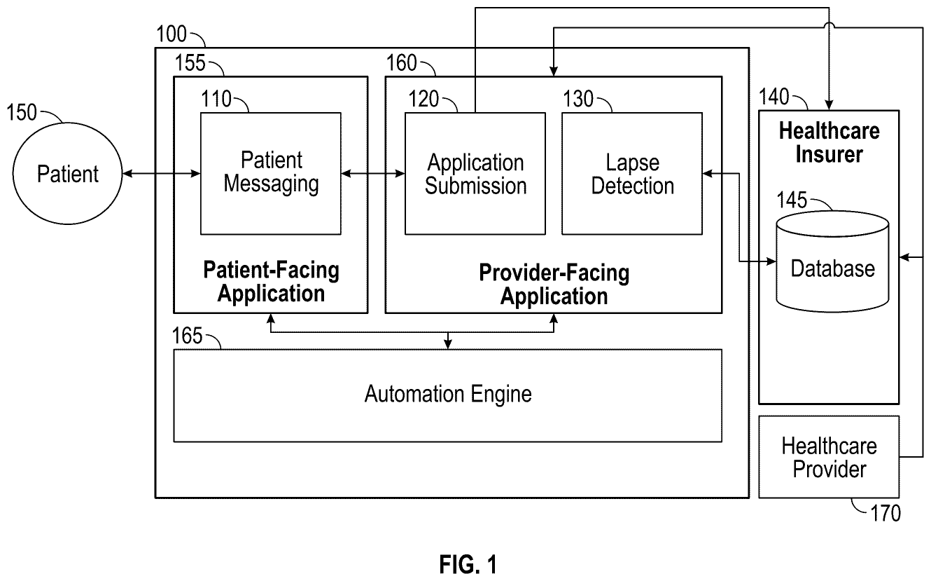

FIG. 1 shows a system 100 of some embodiments for lapse detection. The system 100 includes a patient messaging component 110, an application submission component 120, and a lapse detection component 130. The system 100 is in communication with a healthcare insurer 140, in particular with a database 145 of the healthcare insurer 140. Specifically, in some embodiments such as the example of FIG. 1, the application submission component 120 is in communication with the healthcare insurer 140, and the lapse detection component 130 is in communication with the database 145. The system 100 is also in communication with a patient 150. In some embodiments, the application submission component 120 and/or the lapse detection component 130 are in communication with the healthcare insurer 140 using various protocols, including but not limited to a “270 Request” and “271 Response”, an Application programming Interface (API), or an Electronic Data Interchange (EDI) transaction. Specifically, in some embodiments such as the example of FIG. 1, in some embodiments, the patient messaging component 110 is in communication with the patient 150.

In some embodiments, the patient messaging component 110 is part of a patient-facing application 155, and the application submission component 120 and the lapse detection component 130 are part of a provider-facing application 160. The patient-facing application 155 and the provider-facing application 160 are in communication with each other, to enable data sharing. As an example, the patient messaging component 110 may provide information from the patient 150 to the application submission component 120 which then may provide that information to the healthcare insurer 140. Non-limiting examples of data which can be provided by the patient can include: name, date of birth, address, Social Security number, tax filing information, heath condition, economic condition, household income. In addition, non-limiting examples of documents which the patient may provide can include: proof of identify (e.g., driver's license, passport, birth certificate), proof of income (e.g., pay stub, tax return), proof of residence (e.g., utility bill, rent/mortgage receipt), proof of citizenship status (e.g., I-94, U.S. Citizenship and Immigration Services (USCIS) documentation). The system 100 also includes an automation engine 165 that interfaces with the patient-facing application 155 and the provider-facing application 160 to automate the interactions of the system 100 with the patient 150 and the healthcare insurer 140. The automation engine 165 takes the data received from the patient 150 and prepares it to be submitted as an application for coverage. For example, if a patient's coverage has lapsed, the engine can notify 110 the patient 150 and/or the patient's insurance provider of the lapse, and can provide a unique Uniform Resource Locator (URL) to access the patient-facing web application 155. The patient 150 can then submit updated information to reapply for their coverage. Once the patient 150 submits updated information via the URL, the system prepares 100 and submits the application to the payor on the patient's behalf.

In some embodiments, the lapse detection component 130 detects lapses in medical health insurance coverage for the patient 150 and facilitates re-enrollment with the healthcare insurer 140. For example, some embodiments may include automated checking of source data (e.g., Medicaid enrollment data) to identify when the patient 150 goes from active to inactive coverage status, which is referred to as a lapse. These lapses may be surfaced in the provider-facing application 160. In addition to the application submission component 120 and the lapse detection component 130, the provider-facing application 160 may also provide a user interface (not shown in FIG. 1) for the healthcare insurer 140 or a healthcare provider 170 to display patient data related to health care enrollment and provide automated ways to reach out to the patient 150 (e.g., by email, phone, letter, text message, social media, etc.).

The patient-facing application 155 can be deployed to assist the patient 150 with their application process and may submit an application (or re-application) on the patient's behalf. The patient-facing application 155 may be integrated or interface with the provider-facing application 160 to provide visibility into the application process and resulting data. In addition to the patient messaging component 110, the patient-facing application 155 may include one or more user interfaces (not shown in FIG. 1) for the patient 150, including but not limited to chatbots, online enrollment portals, and virtual assistants. The patient-facing application 155 may be, for example, a user-friendly mobile application that the patient 150 can use to enroll in public coverage with the healthcare insurer 140, submit required documents, and receive assistance with the enrollment process.

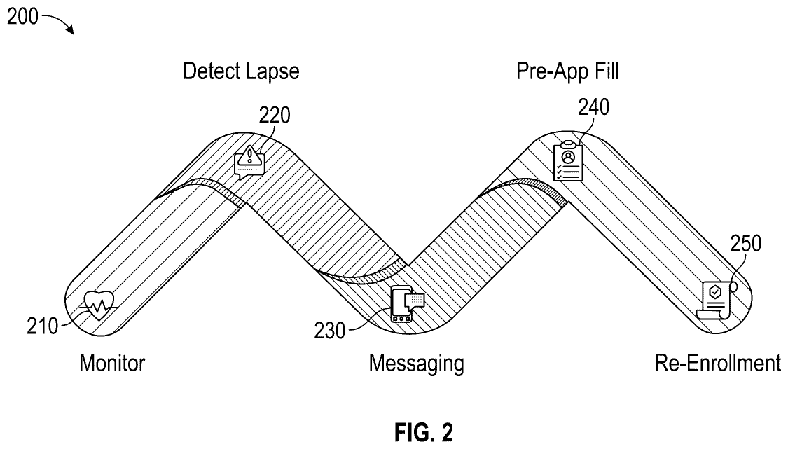

FIG. 2 conceptually shows operations performed in some embodiments by the system 100. The first operation 210 is a monitoring operation, in which the system 100 checks health insurance coverage for applicable patients. As an example, the coverage may be checked on a periodic basis, such as monthly. The first operation 210 may be performed, for example, by the provider-facing application 160, and more specifically may be performed by the lapse detection component 130.

The second operation 220 is a detection of lapses operation, in which the system 100 determines that active coverage has lapsed for a patient. The system 100 may use a custom algorithm to perform lapse detection. The second operation 220 may be performed, for example, by the provider-facing application 160, and more specifically may be performed by the lapse detection component 130.

The third operation 230 is a messaging operation, in which the system 100 informs the patient 150 about the detected lapse in coverage and provides the patient with information about re-enrollment. The third operation 230 may be performed, for example, by the patient-facing application 155, and more specifically may be performed by the patient messaging component 110. The notification of the patient 150 may be facilitated in some embodiments by the automation engine 165.

The fourth operation 240 is a pre-application filling operation, in which the system 100 gathers information need to apply for coverage on the patient's behalf. Patient information can be gathered from one or more sources. 1) Provider: The provider can manually upload a flat file (e.g., Comma Separated Values (CSV)) containing patient identifiable information via the provider-facing web application or Secure File Transfer Protocol (sFTP). Additionally, the provider can automate this process to provide patient data via sFTP or Health Level Seven (HL7). 2) Payor: The identifiable information, insurance subscriber ID, etc., can be received from the payor via the EDI 271 response, depending on the payor's capabilities. 3) Patient: The patient can submit their information through the patient-facing web application when reapplying for coverage. Information from one or all three sources can be used when submitting the final application to the payer. The fourth operation 240 may be performed, for example, by the patient-facing application 155, and more specifically may be performed by the patient messaging component 110.

The fifth operation 250 is a re-enrollment operation, in which the system 100 processes the application and submits the application to the healthcare insurer 140. The application may be processed by a virtual enrollment assistant. The fifth operation 250 may be performed, for example, by the provider-facing application 160, and more specifically the application submission component 120. The application submission component 120 may receive the gathered information from the patient messaging component 110, and then provide the information to the healthcare insurer 140. The application submission may be facilitated in some embodiments by the automation engine 165.

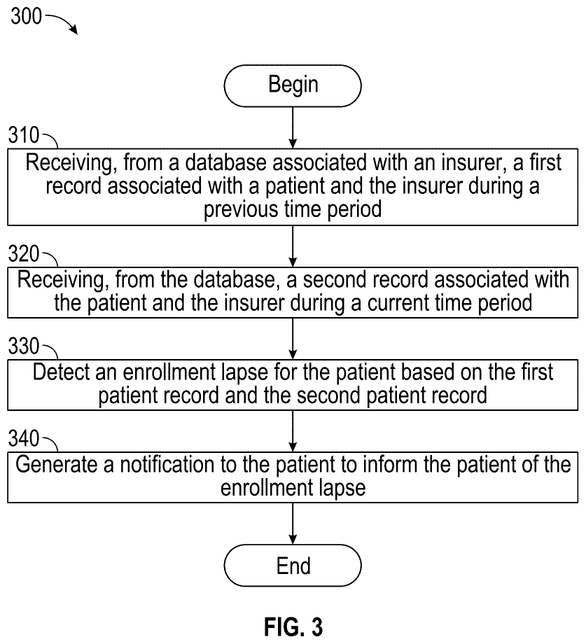

FIG. 3 shows an exemplary process 300 for lapse detection, according to some embodiments. The process 300 may, for example, be performed by the system 100 of FIG. 1.

The process begins at 310 by receiving, from a database (e.g., database 145) associated with a healthcare insurer (e.g., healthcare insurer 140), a first record associated with a patient (e.g., patient 150) and the healthcare insurer during a previous time period. In some embodiments, the process 300 extracts from the first record a prior enrollment status of the patient with the healthcare insurer during the previous time period. For example, the previous time period may be the previous month, or a previous enrollment window.

The process 300 continues at 320 by receiving, from the database, a second record associated with the patient and the healthcare insurer during a current time period. In some embodiments, the process 300 extracts from the second record a current enrollment status of the patient with the healthcare insurer during the current time period. For example, the current time period may be the current month, or a current enrollment window.

In some embodiments, extracting the prior and/or current enrollment status includes transforming the first and second records from a first, proprietary format associated with the database to a second format, such as a format associated with a specific healthcare insurer or state insuring entity. The second format may be, for example, a common format such as an industry standard format. The prior and/or current enrollment status may be extracted from the records in the standard format, after transformation from the proprietary format.

In some embodiments, the first and/or second records are obtained from the database by performing queries to the database, and receiving the records in reply to the queries. The queries may be made on a periodic basis, such as on the first of every month or another period of time corresponding to the enrollment window. Alternatively, the queries may be made in response to a trigger event associated with the patient, such as a change in eligibility, status, employment, etc. In some embodiments, the process 300 performs an authentication with the database to validate an authorization to perform queries.

In some embodiments, operations 310 and/or 320 of process 300 may be performed by the lapse detection component 130 of system 100, which is in communication with the database 145.

The process 300 continues at 330 by detecting an enrollment lapse for the patient based on the first patient record and the second patient record. In some embodiments, the enrollment lapse is detected based on a determination that the prior enrollment status is active and the current enrollment status is inactive.

In some embodiments, operation 330 of process 300 may be performed by the lapse detection component 130 of the system 100. In some embodiments, the lapse detection component 130 uses machine learning algorithms and/or artificial intelligence to predict which patients are at risk of losing their healthcare coverage and proactively reaches out to these patients (e.g., via the patient messaging component 110) to initiate and/or assist with re-enrollment. In some embodiments, the lapse detection component 130 includes algorithms and/or rules-based engines that can identify patterns in public coverage source data (e.g., obtained from the database 145) to detect lapses in coverage for one or more patients.

The process 300 continues at 340 by generating a notification to the patient to inform the patient of the enrollment lapse. The notification may be any one or more of a text message, a phone call, an email, a letter, or a message on social media. In some embodiments, the process 300 also generates a second notification to a medical provider associated with the patient, to inform a healthcare provider associated with the patient (e.g., healthcare provider 170) of the enrollment lapse. In some embodiments, the notifications may be generated by the automation engine 165. The process 300 then ends.

In some embodiments, at 340 the process 300 also changes the current enrollment status of the patient with the healthcare insurer from inactive to active during the current time period. For example, the notification to the patient may include a request for an authorization from the patient and changing the current enrollment status after receiving authorization. The notification may also include access to a patient-facing user interface for the patient to provide enrollment data therethrough to the healthcare insurer to change their enrollment status with the healthcare insurer from inactive to active. The access may be, for example, a link to an online portal for collecting data from the user, or access to an interactive chat interface to guide the user during the enrollment/re-enrollment process. In some embodiments, the notification to the patient may be performed by the patient messaging component 110.

In some embodiments, the patient-facing user interface provides at least a portion of the enrollment data to the healthcare provider and/or the healthcare insurer. In some embodiments, the user interface and data transfer may be provided by the application submission component 120, which is in communication with both the healthcare insurer 140 and the healthcare provider 170.

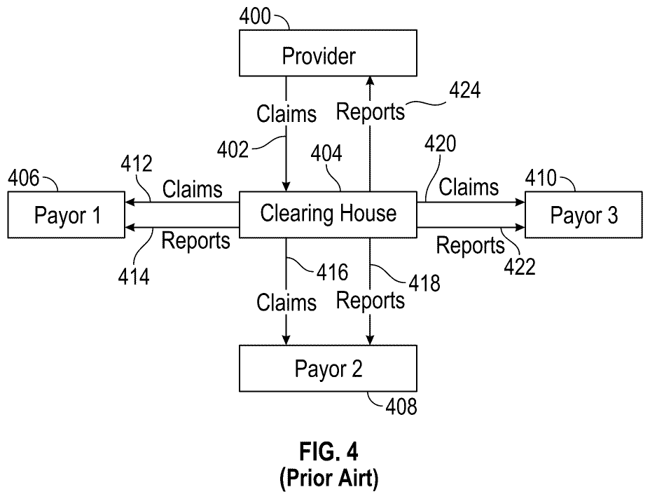

FIG. 4 illustrates an example of communications between a provider, a clearinghouse, and payors. In this example, a provider 400 (such as a doctor, dentist, etc.) sends claims 402 to a clearing house 404. These claims 402 are for services rendered to various patients, and those various patients use different insurance providers (“payors”) 406, 408, 410. The clearing house 404 verifies that the claims 402 are in the correct format and ready to be processed when sent to the various insurance providers/payors 406, 408, 410. The clearing house 404 then sends claims 412, 416, 420 to the respective insurance providers/payors 406, 408, 410, and receives reports 414, 418, 422 indicating if the claims 412, 416, 420 will be paid. The clearing house 404 can then compile these reports 414, 418, 422 and forward the compiled report 424 to the provider 400.

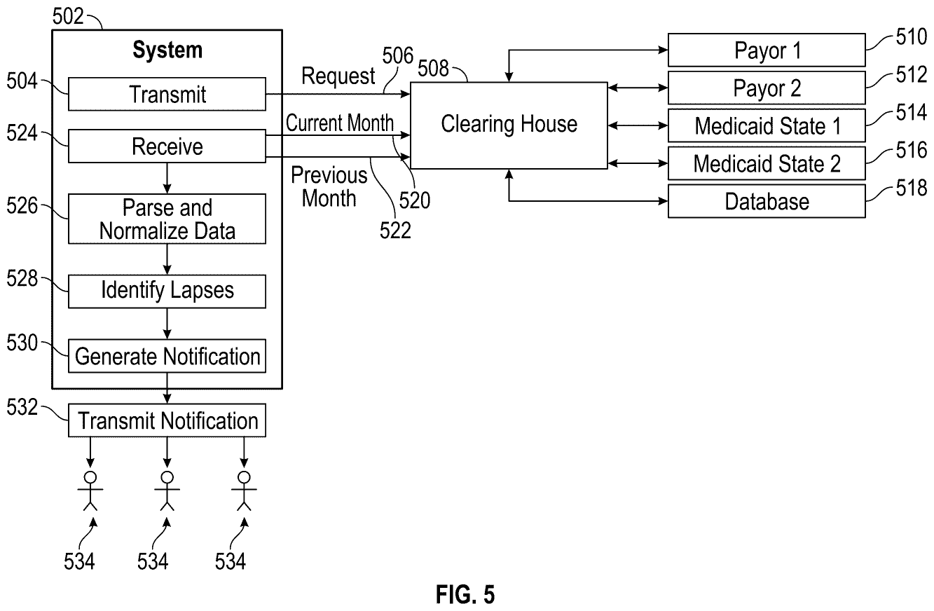

FIG. 5 illustrates an example of a system 502, configured as disclosed herein,

communicating with a clearing house 508, and identifying lapses based on that communication. As illustrated, the system 502 periodically (or upon being triggered) uses transmit 504 communication ability (e.g., a input/output port) to send a request 506 to a clearing house 508 for information about various individuals. Specifically, the request 506 seeks the status of those individuals with regard to their insurance coverage for a current time period (e.g., this month) and the last time period (e.g., last month). The clearing house 508 communicates with one or more payors 512, 514, including state-supported payors such as MEDICAID 514, 516 of one or more states. In some configurations, the clearing house 508 may communicate directly with a database 518 that stores some or all of the data being requested. The respective entities 510, 512, 514, 516, 518 send their data back to the clearing house 508, and the clearing house 508 forwards the data (e.g., the current month 520 enrollment information and the previous month 522 enrollment information) to the system 502, where it is received by a receive 524 portion of the system's communication ability.

Once the system 502 receives the data 520, 522, the system parses and normalizes the data 526. From that parsed/normalized data, the system identifies any lapses 528, generates notifications 530 for those individuals 534, and sends the generated notifications to the individuals 534.

The system may, in some embodiments, include a number of components, each of which may be implemented on a server or on an end-user device. In some cases, a subset of the components may execute on a user device (e.g., a mobile application on a cell phone, a webpage running within a web browser, a local application executing on a personal computer, etc.) and another subset of the components may execute on a server (a physical machine, virtual machine, or container, etc., which may be located at a datacenter, a cloud computing provider, a local area network, etc.).

The components of the system may be implemented in some embodiments as software programs or modules, which are described in more detail below. In other embodiments, some or all of the components may be implemented in hardware, including in one or more signal processing and/or application specific integrated circuits. While the components are shown as separate components, two or more components may be integrated into a single component. Also, while many of the components' functions are described as being performed by one component, the functions may be split among two or more separate components.

In addition, at least one figure conceptually illustrates a process. The specific operations of this process may not be performed in the exact order shown and described. The specific operations may not be performed in one continuous series of operations, and different specific operations may be performed in different embodiments. Furthermore, the process could be implemented using several sub-processes, or as part of a larger macro process.

The terms “light” and “optical” are intended to have broad meanings that can include both visible regions of the electromagnetic spectrum as well as other regions, such as, but not limited to, infrared and ultraviolet light and optical imaging, for example, of such light.

As used in this specification, the terms “computer”, “server”, “processor”, and “memory” all refer to electronic or other technological devices. These terms exclude people or groups of people. As used in this specification, the terms “computer readable medium,” “computer readable media,” and “machine readable medium,” and so forth, are entirely restricted to tangible, physical objects that store information in a form that is readable by a computer. These terms exclude any wireless signals, wired download signals, and any other ephemeral signals.

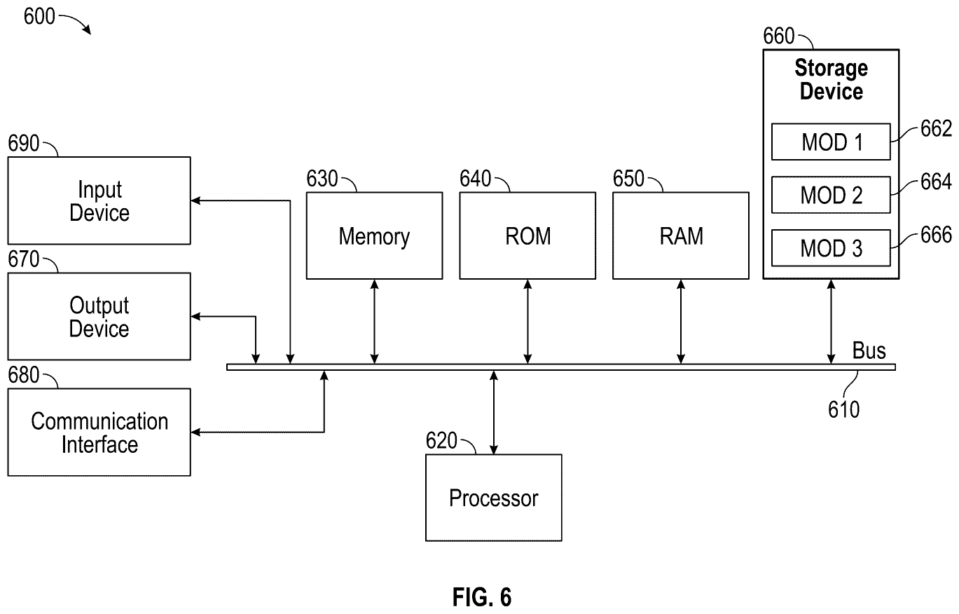

With reference to FIG. 6, an exemplary system includes a computing device 600 (such as a general-purpose computing device), including a processing unit (CPU or processor) 620 and a system bus 610 that couples various system components including the system memory 630 such as read-only memory (ROM) 640 and random access memory (RAM) 650 to the processor 620. The computing device 600 can include a cache of high-speed memory connected directly with, in close proximity to, or integrated as part of the processor 620. The computing device 600 copies data from the system memory 630 and/or the storage device 660 to the cache for quick access by the processor 620. In this way, the cache provides a performance boost that avoids processor 620 delays while waiting for data. These and other modules can control or be configured to control the processor 620 to perform various actions. Other system memory 630 may be available for use as well. The system memory 630 can include multiple different types of memory with different performance characteristics. It can be appreciated that the disclosure may operate on a computing device 600 with more than one processor 620 or on a group or cluster of computing devices networked together to provide greater processing capability. The processor 620 can include any general-purpose processor and a hardware module or software module, such as module 1 662, module 2 664, and module 3 666 stored in storage device 660, configured to control the processor 620 as well as a special-purpose processor where software instructions are incorporated into the actual processor design. The processor 620 may essentially be a completely self-contained computing system, containing multiple cores or processors, a bus, memory controller, cache, etc. A multi-core processor may be symmetric or asymmetric.

The system bus 610 may be any of several types of bus structures including a memory bus or memory controller, a peripheral bus, and a local bus using any of a variety of bus architectures. A basic input/output (BIOS) stored in memory ROM 640 or the like, may provide the basic routine that helps to transfer information between elements within the computing device 600, such as during start-up. The computing device 600 further includes storage devices 660 such as a hard disk drive, a magnetic disk drive, an optical disk drive, tape drive or the like. The storage device 660 can include software modules 662, 664, 666 for controlling the processor 620. Other hardware or software modules are contemplated. The storage device 660 is connected to the system bus 610 by a drive interface. The drives and the associated computer-readable storage media provide nonvolatile storage of computer-readable instructions, data structures, program modules and other data for the computing device 600. In one aspect, a hardware module that performs a particular function includes the software component stored in a tangible computer-readable storage medium in connection with the necessary hardware components, such as the processor 620, system bus 610, output device 670 (such as a display or speaker), and so forth, to carry out the function. In another aspect, the system can use a processor and computer-readable storage medium to store instructions which, when executed by a processor (e.g., one or more processors), cause the processor to perform a method or other specific actions. The basic components and appropriate variations are contemplated depending on the type of device, such as whether the computing device 600 is a small, handheld computing device, a desktop computer, or a computer server.

Although the exemplary embodiment described herein employs the storage device 660 (such as a hard disk), other types of computer-readable media which can store data that are accessible by a computer, such as magnetic cassettes, flash memory cards, digital versatile disks, cartridges, random access memories (RAMs) 650, and read-only memory (ROM) 640, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 600, an input device 690 represents any number of input mechanisms, such as a microphone for speech, a touch-sensitive screen for gesture or graphical input, keyboard, mouse, motion input, speech and so forth. An output device 670 can also be one or more of a number of output mechanisms known to those of skill in the art. In some instances, multimodal systems enable a user to provide multiple types of input to communicate with the computing device 600. The communications interface 680 generally governs and manages the user input and system output. There is no restriction on operating on any particular hardware arrangement and therefore the basic features here may easily be substituted for improved hardware or firmware arrangements as they are developed.

The technology discussed herein refers to computer-based systems and actions taken by, and information sent to and from, computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

Use of language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The embodiments illustrated and discussed in this specification are intended only to teach those skilled in the art how to make and use the technology related to the disclosure. In describing embodiments described herein, specific terminology is employed for the sake of clarity. However, the claims are not to be limited to the specific terminology so selected. The above-described embodiments described herein may be modified or varied, without departing from the scope of the claims, as appreciated by those skilled in the art in light of the above teachings. It is therefore to be understood that, within the scope of the claims and their equivalents, particular embodiments may be practiced otherwise than as specifically described. For example, it is to be understood that the present disclosure contemplates that, to the extent possible, one or more features of any embodiment can be combined with one or more features of any other embodiment.

We claim:

1. A method for lapse detection, comprising:
receiving, at a computer system from a database associated with a healthcare insurer, a first record associated with a patient and the healthcare insurer during a previous time period;
receiving, at the computer system from the database, a second record associated with the patient and the healthcare insurer during a current time period;
detecting, via at least one processor of the computer system, an enrollment lapse for the patient based on the first record and the second record; and
generating, via the at least one processor, a notification to the patient to inform the patient of the enrollment lapse.

2. The method of claim 1, further comprising:
extracting, from the first record, a prior enrollment status of the patient with the healthcare insurer during the previous time period; and
extracting from the second record a current enrollment status of the patient with the healthcare insurer during the current time period,
wherein the enrollment lapse is detected based on a determination that the prior enrollment status is active and the current enrollment status is inactive.

3. The method of claim 2, further comprising changing the current enrollment status of the patient with the healthcare insurer from inactive to active during the current time period.

4. The method of claim 2, wherein the notification comprises a request for an authorization from the patient, the method further comprising changing the current enrollment status after receiving the authorization from the patient.

5. The method of claim 2, further comprising:
transforming the first record from a first format to a second format; and
transforming the second record from the first format to the second format,
wherein the first format is a proprietary format associated with the database, and the second format is a format that is not associated with the database, the prior enrollment status is extracted from the first record in the second format, and the current enrollment status is extracted from the second record in the second format.

6. The method of claim 2, wherein the notification comprises access to a user interface for the patient to provide enrollment data therethrough to the healthcare insurer to change their enrollment status with the healthcare insurer from inactive to active.

7. The method of claim 6, wherein the user interface provides at least a portion of the enrollment data to at least one of a medical provider associated with the patient and the healthcare insurer.

8. The method of claim 1, wherein the previous time period is a month prior to a current month and the current time period is the current month.

9. The method of claim 1, further comprising:
performing a first inquiry to the database; and
performing a second inquiry to the database,
wherein the first record is received from the database in response to the first inquiry and the second record is received from the database in response to the second inquiry.

10. The method of claim 9, wherein at least one of the first inquiry and the second inquiry is performed on a periodic schedule.

11. The method of claim 9, wherein at least one of the first inquiry and the second inquiry is performed based on a trigger event associated with the patient.

12. The method of claim 9, further comprising performing an authentication with the database to validate an authorization to perform queries.

13. The method of claim 1, wherein the notification comprises one of a text message, a phone call, an email, a letter, or a message on social media.

14. The method of claim 1, wherein the notification is a first notification, the method further comprising generating a second notification to a medical provider associated with the patient, to inform the medical provider of the enrollment lapse.

15. A non-transitory computer-readable medium storing a set of instructions for lapse detection, which when executed by a computer, configure the computer to:
receive, from a database associated with a healthcare insurer, a first record associated with a patient and the healthcare insurer during a previous time period;
receive, from the database, a second record associated with the patient and the healthcare insurer during a current time period;
detect an enrollment lapse for the patient based on the first record and the second record; and
generate a notification to the patient to inform the patient of the enrollment lapse.

16. The non-transitory computer-readable medium of claim 15, wherein the instructions, when executed by the computer, further configure the computer to:
extract from the first record a prior enrollment status of the patient with the healthcare insurer during the previous time period; and
extract from the second record a current enrollment status of the patient with the healthcare insurer during the current time period,
wherein the enrollment lapse is detected based on a determination that the prior enrollment status is active and the current enrollment status is inactive.

17. The non-transitory computer-readable medium of claim 16, wherein the instructions, when executed by the computer, further configure the computer to change the current enrollment status of the patient with the healthcare insurer from inactive to active during the current time period.

18. The non-transitory computer-readable medium of claim 16, wherein the notification comprises a request for an authorization from the patient, and
wherein the instructions, when executed by the computer, further configure the computer to change the current enrollment status after receiving the authorization from the patient.

19. The non-transitory computer-readable medium of claim 16, wherein the instructions, when executed by the computer, further configure the computer to:
transform the first record from a first format to a second format; and
transform the second record from the first format to the second format,
wherein the first format is a proprietary format associated with the database, and the second format is a format that is not associated with the database, the prior enrollment status is extracted from the first record in the second format, and the current enrollment status is extracted from the second record in the second format.

20. The non-transitory computer-readable medium of claim 16, wherein the notification comprises access to a user interface for the patient to provide enrollment data therethrough to the healthcare insurer to change their enrollment status with the healthcare insurer from inactive to active.

21. The non-transitory computer-readable medium of claim 20, wherein the user interface provides at least a portion of the enrollment data to at least one of a medical provider associated with the patient and the healthcare insurer.

22. The non-transitory computer-readable medium of claim 15, wherein the previous time period is a month prior to a current month and the current time period is the current month.

23. The non-transitory computer-readable medium of claim 15, wherein the instructions, when executed by the computer, further configure the computer to:
perform a first inquiry to the database; and
perform a second inquiry to the database,
wherein the first record is received from the database in response to the first inquiry and the second record is received from the database in response to the second inquiry.

24. The non-transitory computer-readable medium of claim 23, wherein at least one of the first inquiry and the second inquiry is performed on a periodic schedule.

25. The non-transitory computer-readable medium of claim 23, wherein at least one of the first inquiry and the second inquiry is performed based on a trigger event associated with the patient.

26. The non-transitory computer-readable medium of claim 23, wherein the instructions, when executed by the computer, further configure the computer to perform an authentication with the database to validate an authorization to perform queries.

27. The non-transitory computer-readable medium of claim 15, wherein the notification comprises one of a text message, a phone call, an email, a letter, or a message on social media.

28. The non-transitory computer-readable medium of claim 15, wherein the notification is a first notification, and
wherein the instructions, when executed by the computer, further configure the computer to generate a second notification to a medical provider associated with the patient, to inform the medical provider of the enrollment lapse.
